(19) United States
(12) Patent Application Publication
(10) Pub. No.: US 2011/0009933 A1
(43) Pub. Date: Jan. 13, 2011

(54) PIGGY-BACK PERCUTANEOUS LEAD INSERTION KIT

(75) Inventor: John Michael Barker, Ventura, CA (US)

Correspondence Address: Boston Scientific Neuromodulation Corp.;c/o Frommer Lawrence & Haug LLP, 745 Fifth Ave, NEW YORK, NY, 10151, US

(73) Assignee: Boston Scientific Neuromodulation Corporation, Valencia, CA (US)

(21) Appl. No.: 12/500,447

(22) Filed: Jul. 9, 2009

(51) Int. Cl. A61N 1/05

(52) U.S. Cl. 607/116

(57) ABSTRACT

A kit includes a coupling member and an insertion needle. The coupling member defines at least one lumen extending through the coupling member that is configured and arranged to receive a portion of one or more lead bodies. The insertion needle includes a tubular member that defines a lumen that is optionally configured and arranged to receive a portion of two or more lead bodies that are coupled by a coupling member. A method of implanting a lead comprises coupling together a portion of two or more leads using a coupling member, disposing at least a portion of the two or more leads coupled by the coupling member into a tubular member of an insertion needle, inserting at least the distal end of the tubular member into a tissue of a patient, and advancing the two or more leads coupled by the coupling member distally through the tubular member and into the tissue.

### FIELD

The present invention is directed to the area of implantable electrical stimulation systems and methods of making and using the systems, as well as components of these systems. The present invention is also directed to kits that include coupling members and insertion needles and that may be used to implant leads, such as leads of electrical stimulation systems, as well as methods of implanting leads using coupling members and insertion needles.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One embodiment is a kit comprising a coupling member and an insertion needle. The coupling member defines at least one lumen extending through the coupling member that is configured and arranged to receive a portion of one or more lead bodies. The insertion needle includes a tubular member that defines a lumen extending through the tubular member. The lumen of the tubular member is configured and arranged to receive the distal end of two or more lead bodies that are coupled by a coupling member. The insertion needle may optionally include a beveled tip formed at a distal end of the tubular member. The needle may optionally include a needle hub body coupled to a proximal end of the tubular member. In some embodiments, the needle hub body defines a lumen extending through the needle hub body wherein the lumen is coupled to the proximal end of the lumen extending through the tubular member.

Another embodiment is a method of implanting a lead. The method includes coupling together a portion, such as the distal ends, of two or more leads using a coupling member. The coupling member defines at least one lumen extending through the coupling member. Each of the one or more leads comprising an electrode array disposed on a distal end of the lead body and each electrode array comprising a plurality of electrodes. The method further includes disposing at least a portion, such as the distal ends, of the one or more leads coupled by the coupling member into a lumen of a tubular member of an insertion needle. The insertion needle may optionally include a beveled tip disposed at a distal end of the tubular member. The insertion needle may optionally include a needle hub body coupled to the proximal end of the tubular member. In some embodiments, the needle hub body defines a lumen extending through the needle hub body that is coupled to the lumen extending through the tubular member. The method further includes inserting at least the distal end of the tubular member of the insertion needle into a tissue of a patient. In some embodiments, at least the distal end of the tubular member and the beveled tip of the insertion needle are inserted into a tissue of a patient. The method also includes advancing the distal ends of the two or more leads coupled by the coupling member distally through the tubular member and into the tissue of a patient.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic perspective view of one embodiment of an electrical stimulation system wherein one lead is coupled to a control module, according to the invention;

FIG. 2 is a schematic perspective view of another embodiment of an electrical stimulation system wherein two leads are coupled to a control module, according to the invention;

FIG. 3 is a schematic perspective view of one embodiment of a proximal portion of a lead and a control module of an electrical stimulation system, according to the invention;

FIG. 4 is a schematic perspective view of one embodiment of a proximal portion of a lead, a lead extension and a control module of an electrical stimulation system, according to the invention;

FIG. 5A is a schematic perspective view of one embodiment of the distal ends of two leads coupled by a coupling member, according to the invention;

FIG. 5B is a schematic perspective view of another embodiment of the distal ends of two leads coupled by a coupling member, according to the invention;

FIG. 5C is a schematic perspective view of another embodiment of the distal ends of two leads coupled by a coupling member, according to the invention;

FIG. 5D is a schematic perspective view of another embodiment of the distal ends of two leads coupled by a coupling member, according to the invention;

FIG. 5E is a schematic perspective view of another embodiment of the distal ends of two leads coupled by a coupling member, according to the invention;

FIG. 6 is a schematic perspective view of one embodiment of the distal ends of three leads coupled by two coupling members, according to the invention;

FIG. 7 is a schematic perspective view of one embodiment of a coupling member, according to the invention;

FIG. 8 is a schematic perspective view of another embodiment of a coupling member, according to the invention;

FIG. 9 is a schematic perspective view of another embodiment of a coupling member, according to the invention;

FIG. 10A is a schematic perspective view of one embodiment of an insertion needle, according to the invention;

FIG. 10B is a schematic perspective view of another embodiment of an insertion needle and a syringe, according to the invention;

FIG. 10C is a schematic perspective view of another embodiment of an insertion needle and a syringe, according to the invention;

FIG. 11 is a cross-sectional view of the tubular member of the insertion needle of FIG. 10A at line 11-11;

FIG. 12 is a schematic perspective view of one embodiment of two leads coupled at their distal ends by a coupling member, wherein a steering stylet is inserted into a lumen of one of the leads, according to the invention; and

FIG. 13 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems, as well as components of these systems. The present invention is also directed to kits that include coupling members and insertion needles and that may be used to implant leads, such as leads of electrical stimulation systems, as well as methods of implanting leads using coupling members and insertion needles.

Suitable implantable electrical stimulation systems include, but are not limited to, an electrode lead (“lead”) with one or more electrodes disposed on a distal end of a lead and one or more terminals disposed on one or more proximal ends of the lead. Examples of electrical stimulation systems with leads are found in, for example, U.S. Pat. Nos. 6,181,969; 6,516,227; 6,609,029; 6,609,032; and 6,741,892; and U.S. patent applications Ser. Nos. 10/353,101, 10/503,281, 11/238,240; 11/319,291; 11/327,880; 11/375,638; 11/393,991; and 11/396,309, all of which are incorporated by reference.

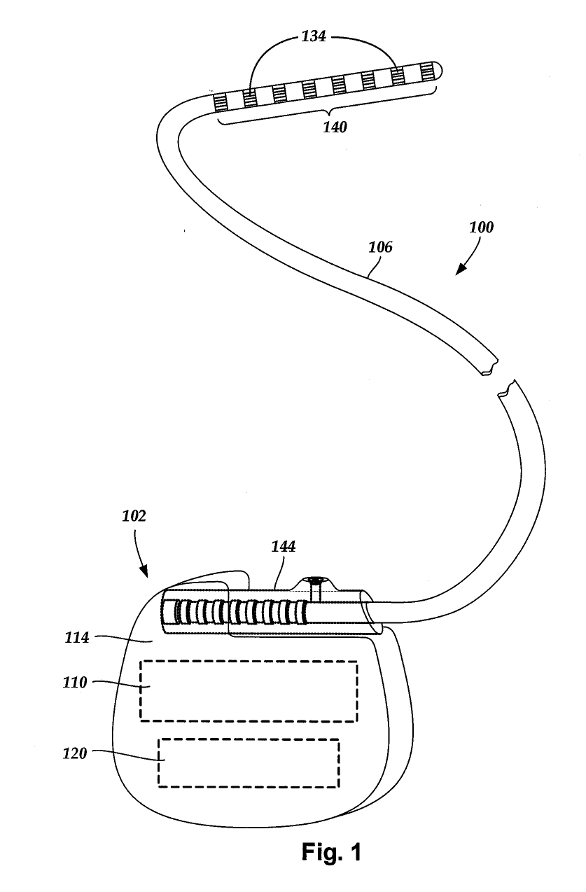

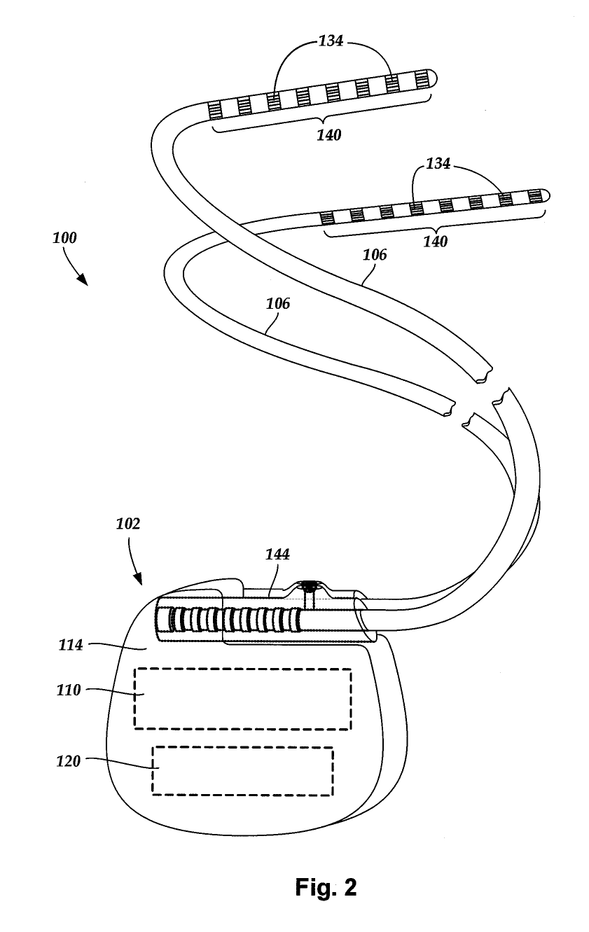

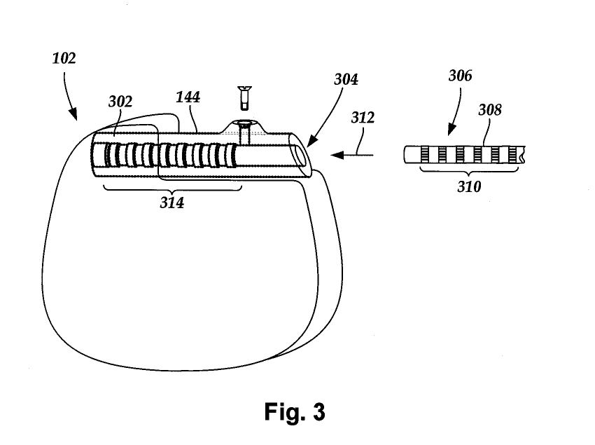

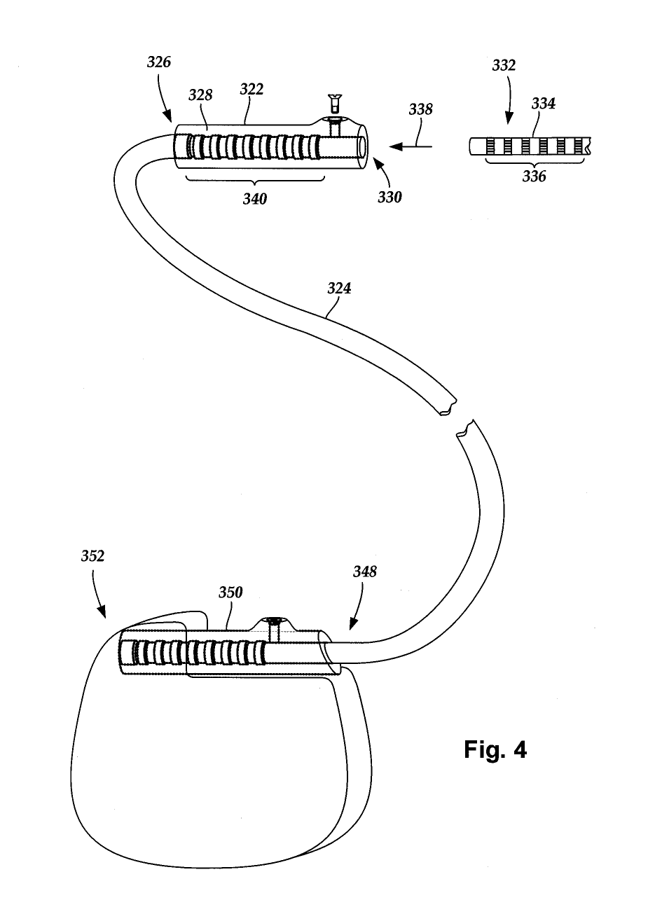

FIG. 1 illustrates schematically one embodiment of an electrical stimulation system 100. The electrical stimulation system includes a control module (e.g., a stimulator or pulse generator) 102 and at least one lead body 106 coupled to the control module 102. Each lead body 106 typically includes an electrode array 140 that comprises at least one electrode 134. The control module 102 typically includes an electronic subassembly 110 and an optional power source 120 disposed in a sealed housing 114. The control module 102 typically includes a connector 144 (FIGS. 1-3; see also 322 and 350 of FIG. 4) into which the proximal end of the one or more lead bodies 106 can be plugged to make an electrical connection via conductive contacts on the control module 102 and terminals (e.g., 310 in FIG. 3 and 336 in FIG. 4) on each of the one or more lead bodies 106. In at least some embodiments, a lead is isodiametric along a longitudinal length of the lead body 106. In addition, one or more lead extensions 324 (see FIG. 4) can be disposed between the one or more lead bodies 106 and the control module 102 to extend the distance between the one or more lead bodies 106 and the control module 102 of the embodiments shown in FIGS. 1 and 2.

As illustrated schematically in FIG. 2, two or more lead bodies 106 can be coupled to the control module 102. The proximal end of each lead body 106 can be plugged into one or more connectors 144 on the control module 102. Each lead body 106 can optionally include an electrode array 140 on the distal end of the lead body 106, wherein each electrode array 140 includes one or more electrodes 134.

The electrical stimulation system or components of the electrical stimulation system, including one or more of the lead bodies 106 and the control module 102, are typically implanted into the body of a patient. The electrical stimulation system can be used for a variety of applications including, but not limited to, brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. The number of electrodes 134 in the electrode array 140 may vary. For example, there can be one, two, three, four, five, six, seven, eight, nine, ten, eleven, twelve, thirteen, fourteen, fifteen, sixteen, or more electrodes 134. As will be recognized, other numbers of electrodes 134 may also be used.

The electrodes 134 of the electrode array 140 are typically disposed in, or separated by, a non-conductive, biocompatible material such as, for example, silicone, polyurethane, polyetheretherketone (“PEEK”), epoxy, and the like or combinations thereof. The lead bodies 106 may be formed in the desired shape by any process including, for example, molding (including injection molding), casting, and the like. The non-conductive material typically extends from the distal end of the one or more lead bodies 106 to the proximal end of each of the one or more lead bodies 106.

Terminals (e.g., 310 in FIG. 3 and 336 in FIG. 4) are typically disposed at the proximal end of the one or more lead bodies 106 of the electrical stimulation system 100 for connection to corresponding conductive contacts (e.g., 314 in FIG. 3 and 340 in FIG. 4) in connectors (e.g., 144 in FIGS. 1-3 and 322 and 350 in FIG. 4) disposed on, for example, the control module 102 (or to conductive contacts on a lead extension, an operating room cable, or an adaptor). Conductor wires (not shown) extend from the terminals (e.g., 310 in FIG. 3 and 336 in FIG. 4) to the electrodes 134. Typically, one or more electrodes 134 are electrically coupled to a terminal (e.g., 310 in FIG. 3 and 336 in FIG. 4). In at least some embodiments, each terminal (e.g., 310 in FIG. 3 and 336 in FIG. 4) is only connected to one electrode 134. The conductor wires may be embedded in the non-conductive material of the lead body 106 or can be disposed in one or more lumens (not shown) extending along the lead body 106. In some embodiments, there is an individual lumen for each conductor wire. In other embodiments, two or more conductor wires may extend through a lumen. There may also be one or more lumens (not shown) that open at, or near, the proximal end of the lead body 106, for example, for inserting a stylet rod to facilitate placement of the lead body 106 within a body of a patient. Additionally, there may also be one or more lumens (not shown) that open at, or near, the distal end of the lead body 106, for example, for infusion of drugs or medication into the site of implantation of the one or more lead bodies 106. In at least one embodiment, the one or more lumens may be flushed continually, or on a regular basis, with saline, epidural fluid, or the like. In at least some embodiments, the one or more lumens can be permanently or removably sealable at the distal end.

In at least some embodiments, leads are coupled to connectors disposed on control modules. In FIG. 3, a lead 308 is shown configured and arranged for insertion into the control module 102. The connector 144 includes a connector housing 302. The connector housing 302 defines at least one port 304 into which a proximal end 306 of a lead 308 with terminals 310 can be inserted, as shown by directional arrow 312. The connector housing 302 also includes a plurality of conductive contacts 314 for each port 304. When the lead 308 is inserted into the port 304, the conductive contacts 314 can be aligned with the terminals 310 on the lead 308 to electrically couple the control module 102 to the electrodes (134 of FIG. 1) disposed at a distal end of the lead 308. Examples of connectors in control modules are found in, for example, U.S. Pat. No. 7,244,150 and U.S. patent application Ser. No. 11/532,844, which are incorporated by reference.

In FIG. 4, a connector 322 is disposed on a lead extension 324. The connector 322 is shown disposed at a distal end 326 of the lead extension 324. The connector 322 includes a connector housing 328. The connector housing 328 defines at least one port 330 into which a proximal end 332 of a lead 334 with terminals 336 can be inserted, as shown by directional arrow 338. The connector housing 328 also includes a plurality of conductive contacts 340. When the lead 334 is inserted into the port 330, the conductive contacts 340 disposed in the connector housing 328 can be aligned with the terminals 336 on the lead 334 to electrically couple the lead extension 324 to the electrodes (134 of FIG. 1) disposed at a distal end (not shown) of the lead 334.

In at least some embodiments, the proximal end of a lead extension is similarly configured and arranged as a proximal end of a lead. The lead extension 324 may include a plurality of conductive wires (not shown) that electrically couple the conductive contacts 340 to a proximal end 348 of the lead extension 324 that is opposite to the distal end 326. In at least some embodiments, the conductive wires disposed in the lead extension 324 can be electrically coupled to a plurality of terminals (not shown) disposed on the proximal end 348 of the lead extension 324. In at least some embodiments, the proximal end 348 of the lead extension 324 is configured and arranged for insertion into a connector disposed in another lead extension. In other embodiments, the proximal end 348 of the lead extension 324 is configured and arranged for insertion into a connector disposed in a control module. As an example, in FIG. 4 the proximal end 348 of the lead extension 324 is inserted into a connector 350 disposed in a control module 352.

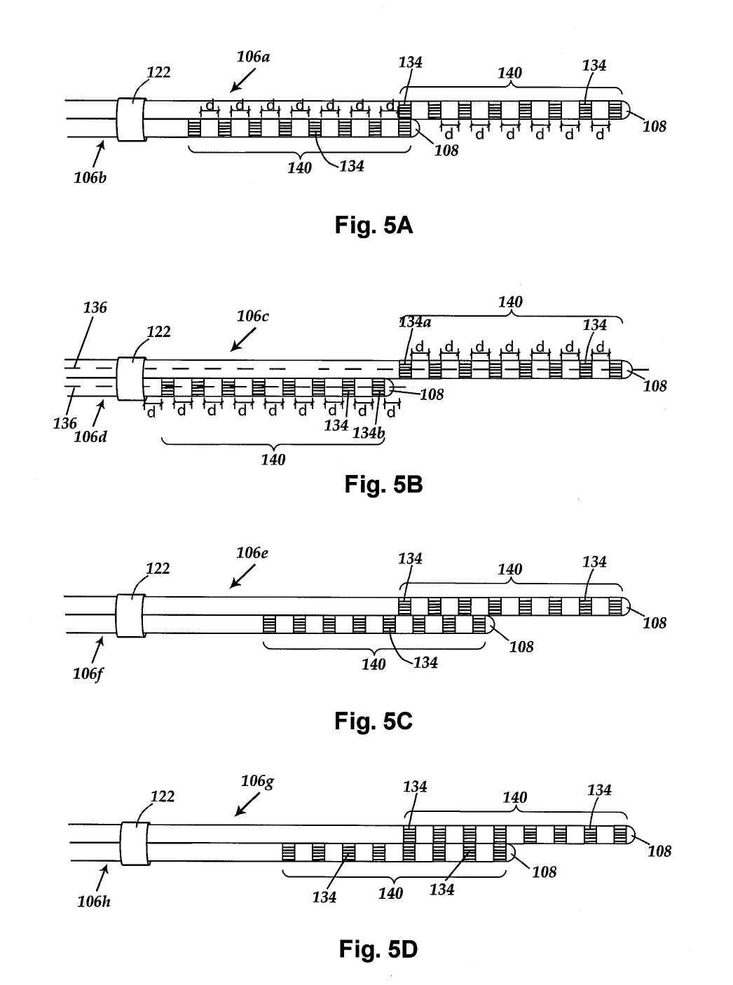

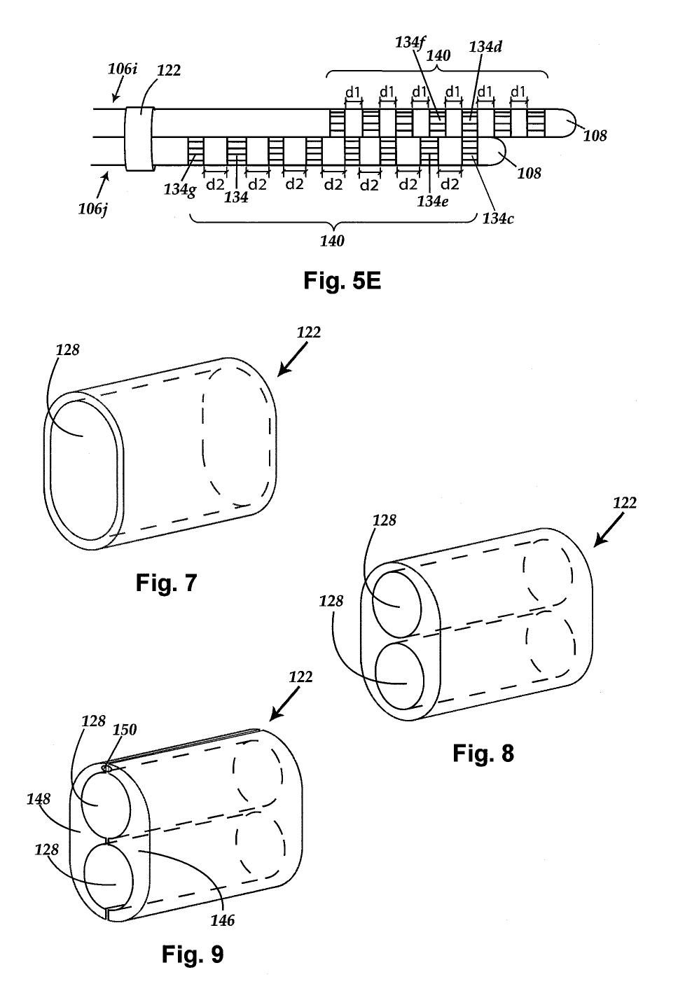

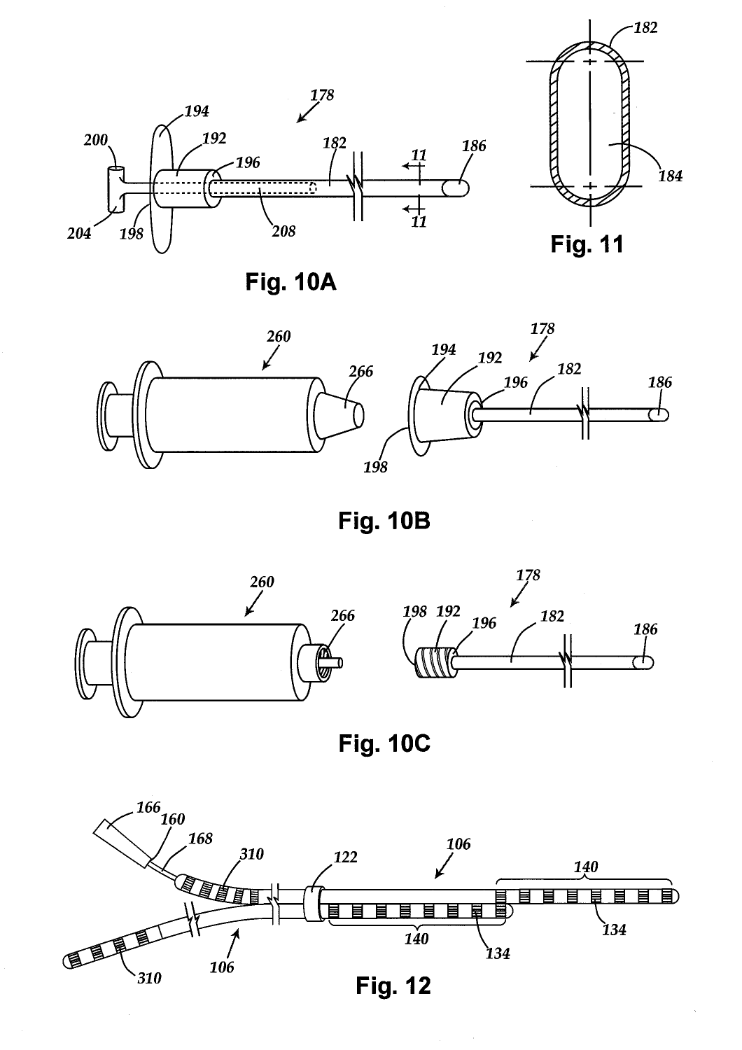

In one embodiment, a kit includes a coupling member 122 (see FIGS. 5A-5E and 6-9) and an insertion needle 178 (see FIGS. 10A, 10B and 10C). In some embodiments, a kit includes a coupling member 122, an insertion needle 178 and two or more leads. In some embodiments, the kits described above may optionally include one or more of a syringe 260 (see FIGS. 10B and 10C), instructions for coupling two or more lead bodies 106 together with a coupling member 122, or instructions for implanting at least the distal ends of two or more lead bodies 106 coupled together with a coupling member 122 into a tissue of a patient using an insertion needle 178. The components of the kits discussed above are described in detail below.

Turning to FIGS. 5A-5E, in some embodiments, at least a portion of two or more lead bodies 106, such as the distal ends of two or more leads, are coupled together by a coupling member 122. The portions of the lead bodies 106 can optionally be coupled such that the distal tips 108 of the lead bodies 106 are staggered as illustrated schematically in FIGS. 5A-5E.

In some embodiments, the portions of the lead bodies 106 are coupled such that the electrode array 140 of a first lead body 106 overlaps with the electrode array 140 of a second lead body as illustrated schematically in FIGS. 5A, 5C, 5D, and 5E. The electrode array 140 includes the most proximal portion of the most proximal electrode 134 to the most distal portion of the most distal electrode 134. For example, the electrode array 140 of lead body 106j in FIG. 5E includes the most proximal portion of electrode 134g to the most distal portion of electrode 134c. In some embodiments, the electrode array 140 of a first lead body 106 does not overlap with the electrode array 140 of a second lead body 106 as illustrated schematically in FIG. 5B.

In some embodiments, the distal ends of two or more lead bodies 106 are coupled by a coupling member 122 such that no electrode 134 disposed on a distal end of a first lead body 106 overlaps with any electrode 134 disposed on a distal end of a second lead body 106 as illustrated schematically in FIGS. 5B and 5C. In some embodiments the electrode array 140 of a first lead body 106 overlaps with the electrode array 140 of a second lead body 106, but no electrode 134 disposed on the distal end of the first lead body 106 overlaps with any electrode 134 disposed on the distal end of the second lead body 106 as illustrated schematically in FIG. 5C. For example, in FIG. 5C, the electrode array 140 of lead body 106e overlaps with the electrode array 140 of lead body 106f, but the electrodes of lead body 106e are not in line with the electrodes of lead body 106f. That is, the electrodes of lead body 106e are staggered with respect to the electrodes of lead body 106f.

In some embodiments, the distal ends of the lead bodies 106 are coupled such that at least one electrode 134 of an electrode array 140 disposed on a distal end of a first lead body 106 overlaps with at least one electrode 134 of an electrode array 140 disposed on a distal end of a second lead body 106 as illustrated schematically in FIGS. 5A, 5D and 5E. A first lead body 106 and a second lead body 106 can optionally be coupled by a coupling member 122 such that zero (see FIGS. 5B and 5C), one (see FIG. 5A), two, three, four (see FIG. 5D), five, six, seven, eight, nine, ten, or more electrodes 134 on the lead body overlap with electrode(s) on the other lead body. In some embodiments, the distal ends of two or more lead bodies 106 are coupled such that some, but not all, of the electrodes 134 disposed on a distal end of a first lead body 106 overlap with one or more electrodes 134 disposed on a distal end of a second lead body 106 as illustrated schematically in FIGS. 5A, 5D and 5E.

The two or more lead bodies 106 can optionally be coupled by a coupling member 122 such that at least one electrode 134 disposed on a first lead body 106 partially, but not fully, overlaps with at least one electrode 134 disposed on a second lead body 106. For example, electrode 134c and electrode 134d in FIG. 5E fully overlap, while electrode 134e and electrode 134f partially, but not fully, overlap.

Electrodes 134 disposed on the distal end of a lead body 106 can be spaced apart at any distance and the distances between electrodes 134 can be uniform or can vary between lead bodies 106 or between electrode 134 pairs on the same lead body 106. In some embodiments, two or more lead bodies 106 are coupled together by a coupling member 122, and electrodes 134 disposed on a distal end of at least one of the lead bodies 106 are separated by equal distances across the electrode array 140 as illustrated schematically in FIG. 5A. For example, in FIG. 5A, all of the electrodes 134 on lead body 106a are separated by a distance d. In some embodiments, all of the electrodes disposed on a first lead body 106 and a second lead body 106 that are coupled by a coupling member 122 are separated by equal distances. For example, in FIG. 5A, all of the electrodes 134 disposed on a first lead body 106a and on a second lead body 106b are separated by a distance d.

In some embodiments, electrodes 134 disposed on a first lead body 106 can be separated by a first distance and electrodes 134 disposed on a second lead body 106 can be separated by a second distance that is different from the first distance. For example, the electrodes 134 disposed on lead body 106i in FIG. 5E are separated by distance d1, while the electrodes 134 disposed on lead body 106j in FIG. 5E are separated by distance d2.

In some embodiments, two or more lead bodies 106 can be coupled by a coupling member 122, wherein the electrodes 134 disposed on each lead body 106 are separated by a distance d. The two or more lead bodies 106 having electrodes 134 separated by a distance d can optionally be coupled such that the most proximally located electrode of a first lead body 106 is also separated longitudinally from the most distally located electrode of a second lead body 106 by a distance d. For example, in FIG. 5B, the electrodes 134 disposed on lead body 106c are separated by a distance d, the electrodes 134 disposed on lead body 106d are separated by a distance d, and lead body 106c and lead body 106d are coupled such that the longitudinal distance between electrode 134a and electrode 134b is distance d. Coupling two or more lead bodies 106 in such a manner will create a uniform stimulation pattern along the combined length of the electrode arrays 140 of lead bodies 106c and 106d.

In some embodiments, a lead axis 136 (see FIG. 5B) of a first lead body 106 is parallel to a lead axis 136 of a second lead body 106 that is coupled to the first lead body 106 by a coupling member. For example, in FIG. 5B, the longitudinal lead axis 136 of lead body 106c is parallel to the longitudinal lead axis 136 of lead body 106d. In some embodiments, the lead axes 136 of three, four, five, six, seven, eight or more lead bodies 106 coupled by one or more coupling members 122 are parallel. In some embodiments, the lead axes 136 of all the lead bodies 106 coupled by one or more coupling members 122 are parallel. In some embodiments, one or more lead bodies 106 are coupled by a coupling member 122 such that the longitudinal axes of each of the coupled lead bodies 106 are parallel, but the longitudinal axes of the lead bodies 106 are not aligned on the same axis.

In some embodiments, a first lead body 106 is coupled to a second lead body 106 by, for example, bonding or thermal joining, before or after the first lead body 106 is coupled to the second lead body 106 by the coupling member 122. As will be recognized, two or more lead bodies 106 can be coupled by, for example, bonding or thermal joining, before or after the two or more lead bodies 106 are coupled by one or more coupling members 122.

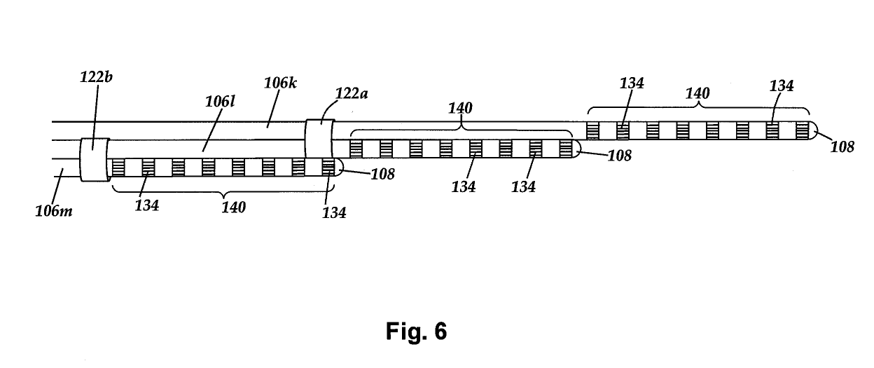

Coupling two or more lead bodies 106 with a coupling member 122 such that the electrode arrays 140 of the two or more lead bodies 106 are staggered longitudinally, for example as illustrated schematically in FIGS. 5A-5E and 6, advantageously allows two or more electrode arrays 140 disposed on two or more lead bodies 106 to be used to form a larger electrode array 140 length. A larger electrode array 140 length advantageously increases the longitudinal stimulation area covered by the two or more lead bodies 106. For example, two lead bodies 106, each having eight electrodes 134 in an electrode array 140, can be coupled by a coupling member 122 to achieve a stimulation pattern equivalent to a lead body 106 with sixteen electrodes 134 in an electrode array 140 as illustrated schematically in FIG. 5B. Therefore, coupling two or more lead bodies 106 with a coupling member 122 allows a practitioner to have more flexibility in designing a stimulation area with one or more types of leads.

Turning to FIG. 6, two or more lead bodies 106 can be coupled by one, two, three, four, five or more coupling members 122. In FIG. 6, lead body 106k is coupled to lead body 106l by coupling member 122a. Lead body 106l is also coupled to lead body 106m by coupling member 122b. Additional coupling members 122 could optionally be used to couple additional lead bodies 106 to lead bodies 106k, 106l or 106m. As will be recognized, lead bodies 106k, 106l, and 106m could optionally be coupled by one coupling member 122.

The coupling members illustrated schematically in FIGS. 5A-5E are disposed around the lead bodies 106 such that the diameter of the lead body 106/coupling member 122 assembly is larger than the diameter of the lead bodies 106 at a location where no coupling member 122 is present. In some embodiments, a portion of a lead body 106 that is bound by a coupling member 122 can have a reduced diameter as compared to at least one other portion of the lead body 106 that is not bound by a coupling member 122. For example, in FIG. 6, a portion of each lead body 106 that has a coupling member 122 disposed around it has a reduced diameter such that when the coupling member 122 is wrapped around that portion, the resulting diameter is equal to the diameter of the remaining portions of the lead body 106 that are not wrapped by a coupling member 122.

Three embodiments of coupling members 122 are illustrated schematically in FIGS. 7, 8 and 9. A coupling member 122 can be made of any biocompatible material such as, for example, polyurethane, silicone, and the like or combinations thereof. A coupling member 122 can be made by any process known to those of skill in the art such as, for example, molding, casting, extruding, and the like.

A coupling member 122 defines at least one lumen 128 extending through the coupling member 122 as illustrated schematically in FIGS. 7, 8 and 9. The coupling member 122 can optionally define one (see FIG. 7), two (see FIGS. 8 and 9), three or more lumens 128. In some embodiments, the number of lumens 128 extending through the coupling member 122 is equal to the number of lead bodies 106 that will be coupled using the coupling member 122.

The at least one lumen 128 of the coupling member 122 is configured and arranged to receive one or more lead bodies 106. For example, a single lumen 128 of a coupling member 122 can optionally be configured and arranged to receive the a portion, such as the distal ends, of two or more lead bodies 106. In one embodiment, a coupling member 122 having one lumen 128 that is configured and arranged to receive two or more lead bodies 106 that are positioned side-by-side is illustrated schematically in FIG. 7. In some embodiments, a coupling member 122 has two lumens 128 that are each configured and arranged to receive one or more lead bodies 106 as illustrated schematically in FIGS. 8 and 9. Although each lumen 128 in the coupling members 122 in FIGS. 8 and 9 are illustrated as being of equal size, it will be recognized that a first lumen 128 in a coupling member 122 can be configured and arranged to receive a different number of lead bodies than a second lumen 128 in the same coupling member 122.

In some embodiments, a coupling member 122 comprises two portions 146, 148. In one embodiment, two portions 146, 148 of a coupling member 122 are illustrated schematically in FIG. 9. In some embodiments, the two portions 146, 148 are entirely separate. For example, the two portions 146,148 can be entirely separate such that they are not permanently coupled, but are coupleable to each other.

In other embodiments, the two portions 146, 148 of the coupling member 122 are joined by a hinge 150 or some other coupling mechanism. In some embodiments, the hinge 50 or other coupling mechanism is located within the body of the coupling member 122 and does not extend beyond the exterior surface of the coupling member 122 as illustrated schematically in FIG. 9.

In some embodiments, at least one of portion 146 or portion 148 includes a locking mechanism that holds portions 146, 148 together when the locking mechanism is engaged. In some embodiments, at least one locking mechanism is configured and arranged to hold portions 146, 148 together such that a lead can be disposed in a lumen formed by portions 146 and 148. In some embodiments, the locking member includes at least one protrusion on one of the portions 146, 148 and at least one corresponding depression on the other of the portions 146, 148. For example, portion 146 can include one or more protrusions that snap into one or more corresponding depressions in portion 148 to hold portions 146 and 148 together.

When two or more lead bodies 106 are coupled using at least one coupling member 122, the at least one coupling member 122 maintains the relative position of at least one of the lead bodies 106 with respect to at least one of the remaining lead bodies 106. For example, the coupling member 122 can optionally maintain the position of a first lead body 106 with respect to the position of a second lead body 106 as illustrated schematically in FIGS. 5A-5E.

Three embodiments of an insertion needle 178 are illustrated schematically in FIGS. 10A, 10B and 10C. An insertion needle 178 includes a tubular member 182 that defines a central lumen 184 (see FIG. 11) extending through the length of the tubular member 182. In some embodiments, the insertion needle 178 is a double-wide needle such that it is configured and arranged to receive two lead bodies 106 arranged side-by-side. In some embodiments, the central lumen 184 is configured and arranged to receive at least a portion, such as the distal ends, of two or more lead bodies 106 that are coupled by a coupling member 122. In some embodiments, the central lumen 184 of the tubular member 182 has a first axis and a second axis, wherein the dimension of the first axis is larger than the dimension of the second axis as illustrated schematically in FIG. 11. In some embodiments, the central lumen 184 of the tubular member 182 has the shape of an ovoid as illustrated schematically in FIG. 11.

In some embodiments, an insertion needle 178 also includes a beveled tip 186 located at a distal end of the tubular member 182 as illustrated schematically in FIGS. 10A, 10B and 10C. The beveled tip 186 is configured and arranged to aid insertion of the insertion needle 178 into tissue of a patient. In some embodiments, the beveled tip 186 is a non-coring beveled tip 186.

In some embodiments, the insertion needle 178 comprises a mating stylet 200 as illustrated schematically in FIG. 10A. The mating stylet 200 includes a handle 204 and a wire 208. The wire 208 of the mating stylet 200 is configured and arranged to slide into the central lumen 184 of the tubular member 182 as illustrated schematically in FIG. 10A.

When an insertion needle 178 with a non-coring beveled tip 186 is inserted into tissue of a patient while the wire 208 of a mating stylet 200 is disposed in the lumen of the tubular member, the non-coring beveled tip 186 and mating stylet 200 will prevent or reduce coring of the tissue such that a portion of the tissue is less likely to become disposed in the central lumen 184 of the tubular member 182.

The tubular member 182 and beveled tip 186 can be made from any biocompatible material that is rigid enough to be inserted into the desired tissue of a patient such as, for example, stainless steel, rigid polymers, and the like or combinations thereof.

In some embodiments, the insertion needle 178 comprises a needle hub body 192. Three embodiments of needle hub bodies 192 are illustrated schematically in FIGS. 10A, 10B and 10C. The needle hub body 192 defines a lumen (not shown) that extends through the length of the needle hub body 192, from the proximal end 198 of the needle hub body 192 to the distal end 196 of the needle hub body 192. The distal end 196 of the needle hub body 192 is coupled to the proximal end of the central lumen 184 of the tubular member 182. The proximal end 198 of the needle hub body 192 may optionally include a needle hub lip 194. Two embodiments of needle hub lips 194 are illustrated schematically in FIGS. 10A and 10B.

A needle hub body 192 can have any shape. In some embodiments, a needle hub body 192 has the shape of a regular or irregular cylinder as illustrated schematically in FIGS. 10A, 10B and 10C. For example, the needle hub body 192 can optionally have the shape of a cylinder with a constant diameter as illustrated schematically in FIGS. 10A and 10C. The needle hub body 192 can optionally have the shape of an irregular cylinder with a varying diameter such that the needle hub body 192 is tapered as illustrated schematically in FIG. 10B. In some embodiments, the needle hub body 192 is configured and arranged to be coupled to a syringe 260, such as a distal portion of a syringe 260. In some embodiments, the lumen of the needle hub body 192 is configured and arranged to receive a syringe 202, such as the distal portion of a syringe 202. For example, the lumen of the needle hub body 192 can optionally be configured and arranged to receive the syringe connector 266. In some embodiments, the needle hub body 192 can be configured and arranged as a female Luer type connector and the distal portion of the syringe 202 can be configured and arranged as a male Luer type connector as illustrated schematically in FIG. 10B.

Either the needle hub body 192 or the distal portion of the syringe 260 can optionally be threaded. For example, the exterior of the needle hub body 192 (see FIG. 10C) or the lumen of the needle hub body 192 can optionally be threaded. The distal portion of the syringe 260, such as the syringe connector 266, can also optionally be threaded. As illustrated schematically in FIG. 10C, in some embodiments, the exterior of the needle hub body 192 is threaded and the syringe connector 266 includes threading that is complementary to the needle hub body 192 threading such that the threading can be used to aid coupling of the needle hub body 192 to the syringe 260. In some embodiments, the interior of the needle hub body 102 is threaded and the syringe connector 266 includes threading that is complementary to the needle hub body 192 threading. In some embodiments, this threaded arrangement is called a Luer lock.

In some embodiments, a method for implanting a lead comprises coupling two or more lead bodies 106 using a coupling member 122. The two or more lead bodies 106 may optionally be coupled by sliding the coupling member 122 over an end, such as a distal end 108, of each lead body 106 to be coupled. For example, two or more lead bodies 106 can optionally be inserted through a single lumen 128 of a coupling member 122 such that the coupling member 122 couples the two or more lead bodies 106. In some embodiments, one or more lead bodies 106 are inserted through each of two or more lumens 128 of a coupling member 122 such that the coupling member 122 couples two or more lead bodies 106.

In some embodiments, a method for implanting a lead comprises disposing one or more lead bodies 106 between two or more portions of a coupling member 122 and then coupling the portions of the coupling member 122 together. For example, one or more lead bodies 106 can be disposed between a first portion 146 and a second portion 148 of a coupling member 122 before the portions 146, 148 are coupled together. In some embodiments, portion 146 and portion 148 are separate. That is, portions 146 and 148 are not permanently coupled.

In some embodiments, a method of implanting a lead comprises disposing one or more lead bodies 106 between portions 146, 148 of a coupling member 122 that are coupled together by a coupling mechanism such as, for example, a hinge 50. The portions 146, 148 of the coupling member 122 can be brought together by, for example, closing a hinge 50 coupling the portions 146, 148 or otherwise engaging a coupling mechanism.

In some embodiments, a method for implanting a lead comprises disposing portion 146 and portion 148 of the coupling member 122 around two or more lead bodies 106 and engaging a locking mechanism to hold portions 146 and 148 together. The locking mechanism can optionally include, for example, a protrusion in one portion and a corresponding depression in the other portion.

The position of the coupling member 122 with respect to the lead bodies 106 can then optionally be adjusted by sliding the coupling member 122 proximally or distally over the lead bodies 106.

In some embodiments, a method of implanting a lead comprises coupling a first lead body 106 to second lead body 106 by, for example, bonding or thermal joining, before or after the first lead body 106 is coupled to the second lead body 106 by the coupling member.

In some embodiments, a method of implanting a lead comprises disposing at least a portion, such as the distal ends, of two or more lead bodies 106 coupled by a coupling member 122 into a central lumen 184 of a tubular member 182 of an insertion needle 178. As described above, the insertion needle 178 may further comprise one or more of a needle hub body 192, a needle hub lip 194, a mating stylet 200, and a beveled tip 186.

In some embodiments, a method of implanting a lead comprises inserting at least the distal end of a tubular member 182 of an insertion needle 178 into a tissue of a patient. The beveled tip 186 and at least the distal end of the tubular member 182 may optionally be inserted into the tissue of the patient. Before the tubular member 182 of the insertion needle 178 is inserted into the tissue of a patient, a mating stylet 200 can be inserted into the lumen 182 of the tubular member 182. As discussed above, the wire 208 of the mating stylet 200 is configured and arranged to slide into the lumen 184 of the tubular member 182 as illustrated schematically in FIG. 10A. In some embodiments, the wire 208 is configured and arranged to fill up the lumen 184 of the tubular member 182 to prevent tissue coring when the tubular member 182 of the insertion needle 178 is inserted into the tissue of a patient.

Either before or after at least the distal end of the tubular member 182 of the insertion needle 178 is inserted into the tissue of a patient, at least a portion, such as the distal ends, of two or more lead bodies 106 coupled by a coupling member 122 can optionally be disposed into the lumen 184 of a tubular member 182 of an insertion needle 178.

After at least the distal end of the tubular member 182 of the insertion needle 178 is inserted into the tissue of a patient, at least a portion, such as the distal ends, of the two or more lead bodies 106 coupled together by a coupling member 122 are advanced distally through the central lumen 184 of the tubular member 182 of the insertion needle 178 and into the tissue of a patient.

In some embodiments, a method of implanting a lead comprises coupling a syringe 260, such as a distal portion of a syringe 260, to the insertion needle 178. For example, the distal portion of the syringe 260 can be coupled to the needle hub body 192 of the insertion needle 178. In some embodiments, a distal portion of a syringe 260 is coupled to an insertion needle 178 by inserting a syringe connector 266 into a lumen of the needle hub body 192. In some embodiments, the needle hub body 192 is configured and arranged as a female Luer type connector and the syringe connector 266 is configured and arranged as a male Luer type connector. In some embodiments, at least a portion of the exterior surface of the needle hub body 192 is threaded and at least a portion of the syringe connector 266 has threading that is complementary to the threading of the needle hub body 192 (see FIG. 10C) such that the threading can be used to aid in coupling the syringe connector 266 and the needle hub body 192.

In some embodiments, the syringe 260 is coupled to the insertion needle 178 after at least the distal ends of two or more lead bodies 106 coupled together by at least one coupling member 122 have been inserted into the central lumen 184 of the tubular member 182 of the insertion needle 178. The syringe 260 coupled to the insertion needle 178 may be used to assist in advancing the coupled lead bodies 106 through the lumen 184 of the tubular member 182 and into the tissue of a patient. For example, the syringe 260 may be utilized by the practitioner for verifying entry into certain tissues or certain regions of the body, such as the epidural space, using, for example, a loss-of-resistance technique.

In some embodiments, a method of implanting a lead comprises inserting one or more steering stylets 160 into at least one lumen of a lead body 106. One embodiment of a steering stylet 160 inserted into the lumen of a lead body 106 is illustrated schematically in FIG. 12. The steering stylet 160 includes a handle 166 and a wire 168 that is configured and arranged to be inserted into a lumen of a lead body 106. For example, the wire 168 of a steering stylet 160 can optionally be inserted into a proximal opening of a lumen of a lead body 106 as illustrated schematically in FIG. 12.

In some embodiments, at least one steering stylet 160 is inserted into a lumen of a lead body 106 that has been coupled to another lead body 106 by a coupling member 122 as illustrated schematically in FIG. 12. The steering stylet 160 may optionally be inserted into the lumen of at least one lead body 106 before or after at least the distal ends of two or more lead bodies 106 coupled together by a coupling member 122 are inserted into the central lumen 184 of the tubular member 182 of an insertion needle 178.

When the wire 168 of the steering stylet 160 is inserted into the lumen of a lead body 106, the steering stylet 160 can be used to aid the practitioner in steering the lead bodies 106 coupled together by the coupling member 122 into a desired location within the tissue of a patient.

In some embodiments, a method of implanting a lead comprises removing the distal end of the tubular member 182, and optionally the beveled tip 186, of the insertion needle 178 from the tissue of a patient after advancing at least the distal end of two or more lead bodies 106 coupled together by a coupling member 122 through the central lumen 184 of the tubular member 182 of the insertion needle 178 and into the tissue of a patient. The distal ends of the two or more lead bodies 106 coupled together by the coupling member 122 are thereby left implanted into the tissue of the patient.

In some embodiments, a method of implanting a lead comprises coupling a proximal end of at least one lead body 106 to a pulse generator. As described above, in some embodiments, the proximal end of a lead body 106 can be inserted into a connector 144 of a control module 102 to make an electrical connection via conductive contacts (e.g., 314 of FIG. 3) on the control module 102 and terminals (e.g., 310 in FIG. 3 and FIG. 12) on each of the one or more lead bodies 106. In at least some embodiments, a proximal end of at least one lead body 106 is coupled to a pulse generator after the distal end of two or more lead bodies 106 coupled together by a coupling member 122 are inserted into the tissue of a patient.

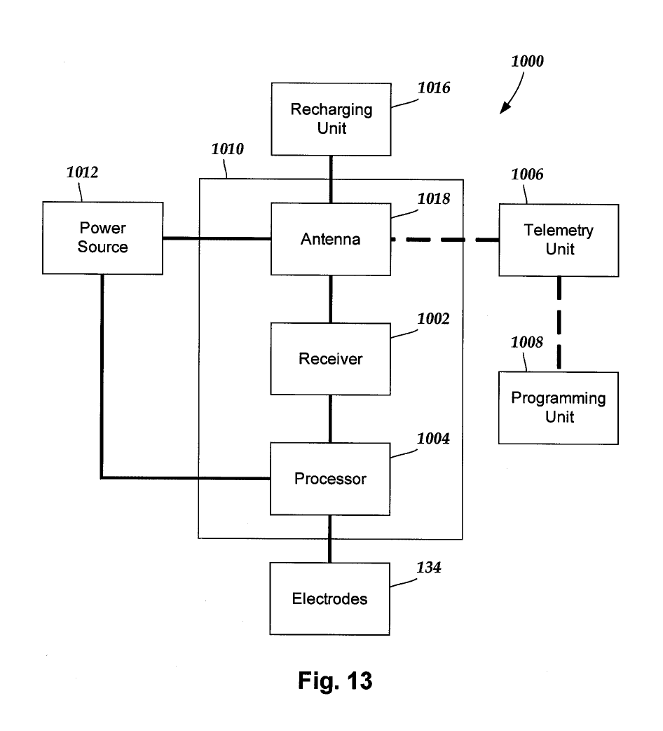

FIG. 13 is a schematic overview of one embodiment of components of an electrical stimulation system 1000 including an electronic subassembly 1010 disposed within a control module. It will be understood that the electrical stimulation system can include more, fewer, or different components and can have a variety of different configurations including those configurations disclosed in the stimulator references cited herein.

Some of the components (for example, power source 1012, antenna 1018, receiver 1002, and processor 1004) of the electrical stimulation system can be positioned on one or more circuit boards or similar carriers within a sealed housing of an implantable pulse generator, if desired. Any power source 1012 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Patent Application Publication No. 2004/0059392, incorporated herein by reference.

As another alternative, power can be supplied by an external power source through inductive coupling via the optional antenna 1018 or a secondary antenna. The external power source can be in a device that is mounted on the skin of the user or in a unit that is provided near the user on a permanent or periodic basis.

If the power source 1012 is a rechargeable battery, the battery may be recharged using the optional antenna 1018, if desired. Power can be provided to the battery for recharging by inductively coupling the battery through the antenna to a recharging unit 1016 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. A processor 1004 is generally included to control the timing and electrical characteristics of the electrical stimulation system. For example, the processor 1004 can, if desired, control one or more of the timing, frequency, strength, duration, and waveform of the pulses. In addition, the processor 1004 can select which electrodes can be used to provide stimulation, if desired. In some embodiments, the processor 1004 may select which electrode(s) are cathodes and which electrode(s) are anodes. In some embodiments, the processor 1004 may be used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1008 that, for example, allows modification of pulse characteristics. In the illustrated embodiment, the processor 1004 is coupled to a receiver 1002 which, in turn, is coupled to the optional antenna 1018. This allows the processor 1004 to receive instructions from an external source to, for example, direct the pulse characteristics and the selection of electrodes, if desired.

In one embodiment, the antenna 1018 is capable of receiving signals (e.g., RF signals) from an external telemetry unit 1006 which is programmed by a programming unit 1008. The programming unit 1008 can be external to, or part of, the telemetry unit 1006. The telemetry unit 1006 can be a device that is worn on the skin of the user or can be carried by the user and can have a form similar to a pager, cellular phone, or remote control, if desired. As another alternative, the telemetry unit 1006 may not be worn or carried by the user but may only be available at a home station or at a clinician's office. The programming unit 1008 can be any unit that can provide information to the telemetry unit 1006 for transmission to the electrical stimulation system 1000. The programming unit 1008 can be part of the telemetry unit 1506 or can provide signals or information to the telemetry unit 1006 via a wireless or wired connection. One example of a suitable programming unit is a computer operated by the user or clinician to send signals to the telemetry unit 1006.

The signals sent to the processor 1004 via the antenna 1018 and receiver 1002 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1000 to cease operation, to start operation, to start charging the battery, or to stop charging the battery. In other embodiments, the stimulation system does not include an antenna 1018 or receiver 1002 and the processor 1004 operates as programmed.

Optionally, the electrical stimulation system 1000 may include a transmitter (not shown) coupled to the processor 1004 and the antenna 1018 for transmitting signals back to the telemetry unit 1006 or another unit capable of receiving the signals. For example, the electrical stimulation system 1000 may transmit signals indicating whether the electrical stimulation system 1000 is operating properly or not or indicating when the battery needs to be charged or the level of charge remaining in the battery. The processor 1004 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A kit comprising:
a coupling member defining at least one lumen extending through the coupling member, wherein the at least one lumen is configured and arranged to receive a portion of one or more lead bodies; and
an insertion needle comprising:
a tubular member defining a lumen extending through the tubular member, wherein the lumen of the tubular member is configured and arranged to receive the distal end of two or more lead bodies that are coupled by a coupling member;
a beveled tip formed at a distal end of the tubular member; and
a needle hub body coupled to a proximal end of the tubular member, wherein the needle hub body defines a lumen extending through the needle hub body, and wherein the needle hub body lumen is coupled to the proximal end of the lumen extending through the tubular member.

2. The kit of claim 1, wherein the coupling member defines exactly one lumen, and wherein the lumen is configured and arranged to receive at least a portion of two or more lead bodies.

3. The kit of claim 1, wherein the coupling member defines two lumens and wherein each of the two lumens is configured and arranged to receive at least a portion of one or more lead bodies.

4. The kit of claim 1, further comprising a first lead and a second lead, wherein each lead comprises a lead body and an electrode array disposed on a distal end of the lead body; wherein each electrode array comprises a plurality of electrodes.

5. The kit of claim 4, wherein the coupling member couples the first lead to the second lead at a distal end of each lead.

6. The kit of claim 5, wherein the first lead is coupled to the second lead via the coupling member such that at least one electrode of the electrode array of the first lead overlaps at least one electrode of the electrode array of the second lead.

7. The kit of claim 5, wherein the first lead is coupled to the second lead via the coupling member such that the electrode array of the first lead does not overlap the electrode array of the second lead.

8. The kit of claim 5, wherein the first lead is coupled to the second lead via the coupling member such that no electrode of the electrode array of the first lead overlaps any electrode of the electrode array of the second lead.

9. The kit of claim 5, wherein a portion of the first lead body and a portion of the second lead body that are disposed within the lumen of the coupling member have a reduced diameter as compared to other portions of the first lead body and the second lead body not disposed in the lumen of the coupling member.

10. The kit of claim 1, wherein the needle hub body is configured and arranged to be coupled to a syringe.

11. The kit of claim 1, wherein the insertion needle further comprises a mating stylet comprising a wire and a handle, and wherein the wire is configured and arranged to be inserted into the lumen of the tubular member.

12. The kit of claim 1, wherein the lumen of the tubular member has the shape of an ovoid.

13. A method of implanting a lead comprising:
coupling together distal ends of two or more leads using a coupling member, wherein the coupling member defines at least one lumen extending through the coupling member, wherein each lead comprises an electrode array disposed on a distal end of a lead body, and wherein each electrode array comprises a plurality of electrodes;
disposing at least the distal ends of the two or more leads coupled by the coupling member into a lumen of a tubular member of an insertion needle, wherein the insertion needle further comprises a beveled tip disposed at a distal end of the tubular member, and a needle hub body coupled to the proximal end of the tubular member, wherein the needle hub body defines a lumen extending through the needle hub body that is coupled to the lumen extending through the tubular member; and
inserting the beveled tip and at least the distal end of the tubular member of the insertion needle into a tissue of a patient; and
advancing the distal ends of the two or more leads coupled by the coupling member distally through the tubular member and into the tissue of the patient.

14. The method of implanting a lead of claim 13, wherein coupling a distal end of two or more leads using a coupling member comprises coupling the distal end of a first lead to the distal end of a second lead such that the electrode array disposed on the distal end of the first lead does not overlap the electrode array disposed on the distal end of the second lead.

15. The method of implanting a lead of claim 13, wherein coupling a distal end of two or more leads using a coupling member comprises coupling the distal end of a first lead to the distal end of a second lead such that the electrode array disposed on the distal end of the first lead at least partially overlaps the electrode array disposed on the distal end of the second lead.

16. The method of implanting a lead of claim 13, wherein the plurality of electrodes in each electrode array are separated by the same distance, and wherein coupling a distal end of two or more leads using a coupling member comprises coupling the distal end of a first lead to the distal end of a second lead such that the longitudinal distance between the most proximal electrode disposed on the first lead and the most distal electrode disposed on the second lead is equal to the distance between the electrodes in each electrode array.

17. The method of implanting a lead of claim 13, further comprising attaching a syringe to the needle hub body after disposing at least the distal ends of the two or more leads coupled by the coupling member into the lumen of the tubular member.

18. The method of implanting a lead of claim 13, further comprising:
inserting a steering stylet into a proximal end of at least one lumen disposed within at least one of the two or more leads; and
using the steering stylet to position at least one distal end of the two or more leads coupled by the coupling member into a desired location within the tissue of the patient.

19. The method of implanting a lead of claim 13, further comprising removing the beveled tip and the distal end of the tubular member of the insertion needle from the tissue of the patient after advancing the distal ends of the two or more leads coupled by the coupling member distally through the tubular member and into the tissue of the patient, thereby leaving the distal ends of the two or more leads and the coupling member implanted in the tissue of the patient.

20. The method of implanting a lead of claim 13, further comprising coupling a proximal end of the two or more leads to a pulse generator after advancing the distal ends of the two or more leads coupled by the coupling member distally through the tubular member and into the tissue of the patient.

21. The method of implanting a lead of claim 13, wherein the lumen of the tubular member has the shape of an ovoid.
